(19) United States
(12) Patent Application Publication
(10) Pub. No.: US 2017/0049654 A1
(43) Pub. Date: Feb. 23, 2017

(54) ASSEMBLY AND SYSTEM FOR DISTRACTOR AND/OR POSITIONER

(71) Applicant: Innovative Medical Products, Inc., Plainville, CT (US)

(72) Inventors: Edward DEMAYO, San Rafael, CA (US); Tim BLACKWELL, Jupiter, FL (US); Tamas KOVACS, Bristol, CT (US)

(73) Assignee: Innovative Medical Products, Inc., Plainville, CT (US)

(21) Appl. No.: 15/048,577

(22) Filed: Feb. 19, 2016

Related U.S. Application Data
(60) Provisional application No. 62/118,009, filed on Feb. 19, 2015.

(51) Int. Cl. A61G 13/12; A61B 17/66; A61F 5/37

(52) U.S. Cl. CPC A61G 13/1245; A61F 5/3761; A61B 17/66; A61G 13/127

(57) ABSTRACT

A sterile pressure pad assembly system, kit and method uses a cage assembly comprising an enclosure and a pad assembly configured as a generally rectangular pad disposed adjacent the patient's limb near the joint to be extended and/or distended. The pad may be affixed by adhesive to the outer surface of an arcuate side and formed from suitable foam and cell structure materials that can be sterilized such as, for example, polyurethane-based foam. The enclosure of the cage assembly is configured with one or more ridges to sufficiently optimize a material strength to the arcuate side of the enclosure so as to not crush under pressure from the surgical distractor unit and/or the weight of the patient's limb of the patient when the sterile cage assembly and pad support a limb of a patient on a support bar as a surgical distractor unit positions the pad in abutment with a popliteal area of a patient's knee joint during a medical procedure.

### CROSS-REFERENCE TO RELATED APPLICATION

This application claims the benefit of U.S. Provisional Application No. 62/118,009 filed Feb. 19, 2016 for “Assembly And System For Distractor And/Or Positioner,” which is incorporated herein by reference in its entirety.

### FIELD OF THE INVENTION

The present invention relates to reducing the tissue trauma that occurs when a physician distends or otherwise deforms a patient's tissues in a surgical operation or for other medical procedures and, more particularly, to an improved positioning pad assembly and system for securing and holding a limb in a particular position and/or in conjunction with distraction of a joint, bone that advantageously reduces slippage and is particularly useful in the positioning of a larger, heavier body part.

### BACKGROUND OF THE INVENTION

Conventional positioning and distraction systems are useful in positioning a body part (e.g. limb, knee, bone, joint, etc.) of a patient for robotic, laparoscopic and other medical procedures when a physician distends or otherwise deforms a patient's tissues in a surgical operation. For example, distraction may be used in medical procedures to separate two parts of a bone after the bone is transected. Also, distraction may be used in surgical procedures to provide distraction of joint surfaces without displacement and/or rupture of their binding ligaments. Consequently, these endoscopic, robotic, laparoscopic and other medical procedures use surgical distraction systems to separate bones, bony fragments or joint surfaces of a limb, e.g. when such extension is desired.

Conventional surgical positioning and distraction systems may use a foam pad and/or a cohesive wrap to protect a patient from pressure sores, abrasions and possible neurological impairment while securing foot into the boot. For example, sterile foam pads and wraps may be utilized to with surgical distraction systems for reducing the pressure placed against the thigh in a medical procedure on a knee or ankle (e.g. pressure sores, abrasions and possible neurological impairment). In such medical procedures, a foam pad and/or wrap also must be sterilized and are typically single use.

Conventional sterile foam pads have problems in maintaining position on a bracket and/or in a boot device as these may slip or shift when a distractor is used for flexion, extension, tilt, and rotation, e.g. when distracting the ankle or knee during arthroscopic surgery. Such problems in conventional wraps and foam pads are amplified when heavier, larger limbs of a heavier and/or larger patient are being positioned for surgery as well as during surgery. One solution has been to increase the pressure on the pad using distraction; however, pads slide off of the support post, fail and any such increased pressure can be harmful to the circulatory system, tissues, bones, and patient health. As a result, there is a need for an improved positioning pad assembly and system for securing and holding a limb in a particular position and/or in conjunction with distraction of a joint, bone or in the positioning of a body part for a surgical operation to overcome these and other harmful problems to the circulatory system, tissues, bones, and patient health that are present in conventional surgical positioning and distraction systems.

The present invention solves these problems with a pad assembly and system that has improved holding, resists slippage, and results in less harm to the patient's circulatory system, tissues, and/or bones, thereby improving a patient's health. The improved pad assembly and system according to the present invention maintains its hold and position on a support bar or distractor bar bracket, thereby allowing reduced applied pressure of the distractor against the thigh as compared to conventional surgical positioning and distraction systems. Moreover, the present invention reduces repositioning because its design reduces support bar sliding and slippage especially when distracting heavier, larger limbs of a heavier and/or larger patient before and during a surgical procedure.

### SUMMARY OF THE INVENTION

It is an object of the present invention to provide a pad assembly, system, kit, and method of positioning and holding a foam pad assembly and system that reduces and/or eliminates harmful problems to the circulatory system, tissues, bones, patient health, and otherwise is gentler for the patient.

An object of the present invention is to provide a pad assembly and system for securing and holding a limb in a particular position and/or in conjunction with the positioning of a body part, distraction of a joint, and distraction of bone in a medical procedure.

Another object of the present invention is to provide a protector pad assembly and system for reducing harm to the circulatory system, tissues, bones, and improving patient health by distributing pressure over a large area of the distal femur reducing the direct application of pressure on tissues, vessels and the popliteal nerve.

Yet another object of the present invention is to provide a protector pad assembly and system that maintains its position so as not to shift, slide or roll into the popliteal area. It is an object of the present invention to provide these advantages for a pad assembly that overcomes the shifting into the popliteal area and related problems that are amplified when larger, heavier limbs are being positioned for surgery and throughout a surgical procedure.

Still yet another object of the present invention is to provide a protector pad assembly and system with advantages of a sterilized product that may be produced and sold as a pressure protector pad set or kit for use with a positioning and/or distractor system such as, for example, the De Mayo Universal Distractor as disclosed in U.S. Pat. No. 8,048,082 B1.

It is an object of the present invention to provide an apparatus, system and method of positioning and holding a sterile pad assembly and system with design features for locking to a support bar so as to hold and maintain its position thereby reducing pressure needed to be placed against the thigh in a typical procedure without repositioning.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Description of the Embodiments, which is to be read in association with the accompanying drawings, which are incorporated in and constitute a part of this specification, show certain aspects of the subject matter disclosed herein and, together with the description, help explain some of the principles associated with the disclosed implementations, wherein:

FIG. 1 illustrates is a perspective view of the apparatus system in accordance with an embodiment of the present invention;

FIG. 2 illustrates is a side view of the apparatus and system;

FIG. 3 illustrates is an end view of the apparatus and system;

FIG. 4 illustrates is a top view of the apparatus and system;

FIG. 5 illustrates is a bottom view of the apparatus and system;

FIG. 6A illustrates a bottom view, FIG. 6B illustrates a side view, and FIG. 6C illustrates a sectional view of the apparatus and system along circle line C-C in accordance with an embodiment of the present invention;

FIG. 7A illustrates a side view of the side portion and opening taken along lines A-A of FIG. 6A; and 7B illustrate is sectional view along circle line B-B of FIG. 7A of the apparatus and system in accordance with an embodiment of the present invention;

FIG. 8 illustrates a perspective view of the foam protector pad;

FIG. 9 illustrates an end view of the foam protector pad;

FIG. 10 illustrates a top and or bottom view of the foam protector pad;

FIG. 11 illustrates an end view of the foam protector pad bend to shape in accordance with an embodiment of the present invention;

FIG. 12 illustrates is a perspective view of the foam protector pad bend to shape;

FIG. 13 illustrates is a side view of the foam protector pad bend to shape;

FIG. 14 illustrates is a side view of support of the pad assembly and system in accordance with an embodiment of the present invention

FIG. 15 illustrates is a bottom view of support of the pad assembly and system

FIG. 16 illustrates is an end view of the support of the pad assembly and system;

FIG. 17 illustrates is a side perspective view of the support the pad assembly and system in a surgical operation with a distractor; and

FIG. 18 illustrates is an anatomical view of the posterior of a human limb.

### DESCRIPTION OF THE EMBODIMENTS

Non-limiting embodiments of the present invention will be described below with reference to the accompanying drawings, wherein like reference numerals represent like elements throughout. While the invention has been described in detail with respect to the preferred embodiments thereof, it will be appreciated that upon reading and understanding of the foregoing, certain variations to the preferred embodiments will become apparent, which variations are nonetheless within the spirit and scope of the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “some embodiments”, “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are provided for the purposes of illustrating some embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the term “popliteal area” refers to the posterior area of the human leg around the knee joint and its tissues, bones circulatory and nerve structures. The area is visually identifiable as a shallow depression located at the back of the knee joint that includes the popliteal artery, popliteal nerve, tibial artery, tibial nerve and other tissues.

As used herein the term “popliteal artery” refers to an artery that is a deeply placed continuation of the femoral artery after it passes through the adductor hiatus, or opening in the distal portion of the adductor magnus muscle. The popliteal artery courses through the popliteal fossa and ends at the lower border of the popliteus muscle, where it branches into the anterior and posterior tibial arteries.

As used herein the term “popliteal nerve” refers to a common fibular nerve (common peroneal nerve; external popliteal nerve; peroneal nerve; lateral popliteal nerve), about one-half the size of the tibial nerve, is derived from the dorsal branches of the fourth and fifth lumbar and the first and second sacral nerves. The popliteal nerve descends obliquely along the lateral side of the popliteal fossa to the head of the fibula, close to the medial margin of the biceps femoris muscle and the common peroneal nerve winds round the head of the fibula as is illustrated in FIG. 18.

As used herein the term “tibial artery” refers to the anterior tibial artery of the leg functioning to carry blood to the anterior compartment of the leg and dorsal surface of the foot, from the popliteal artery, which also is accompanied the anterior tibial vein, along its course.

As used herein the term “tibial nerve” refers to the tibial nerve that is a branch of the sciatic nerve. The tibial nerve passes through the popliteal fossa to pass below the arch of soleus.

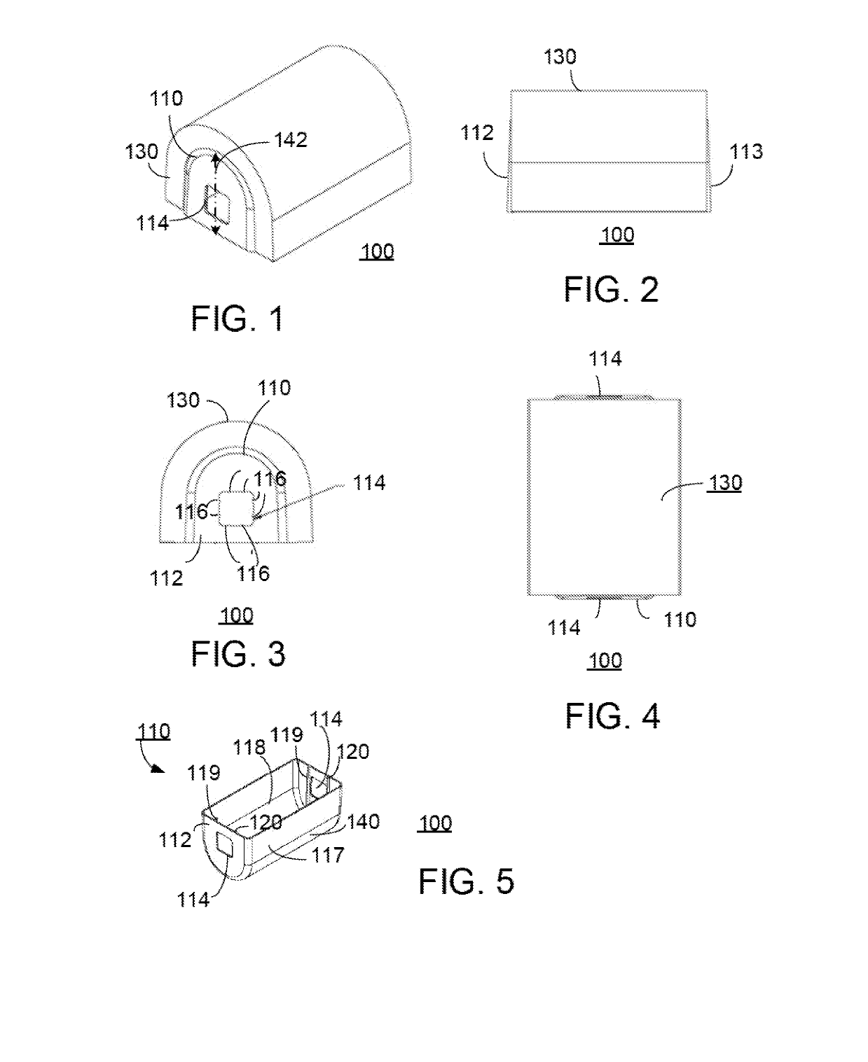

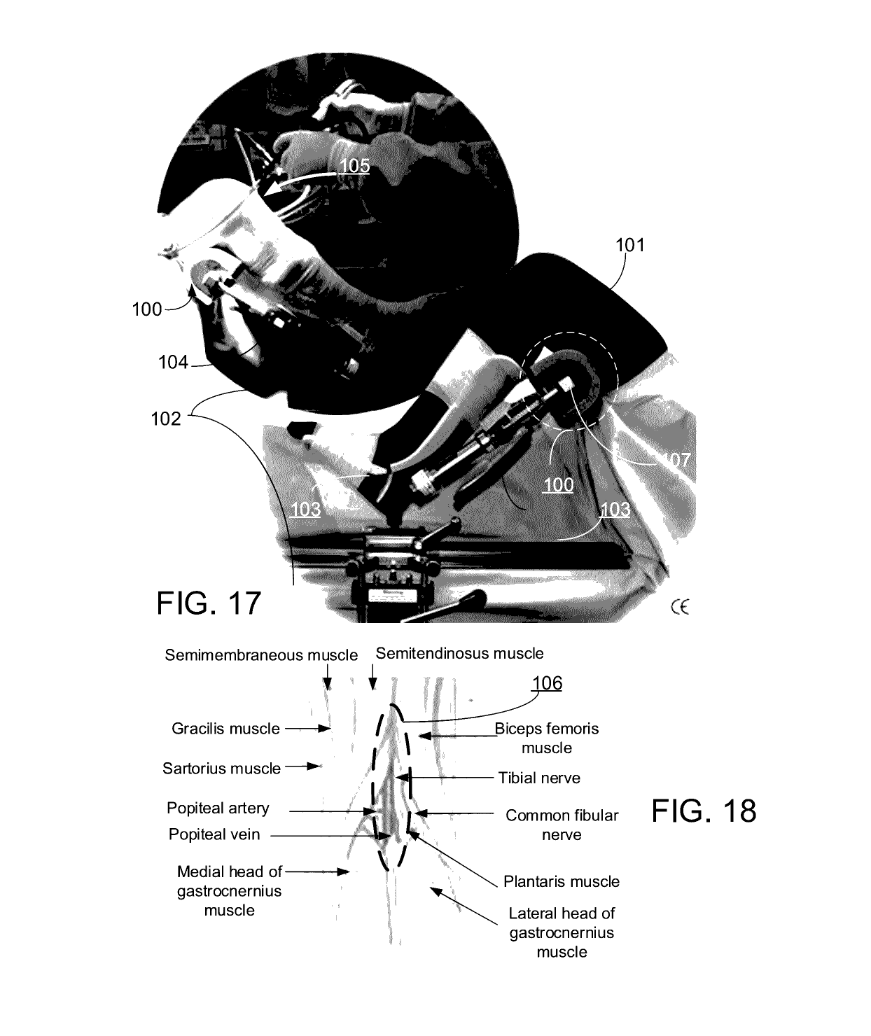

As is illustrated in FIGS. 1-18, the pressure pad apparatus and system 100, such as shown in FIG. 1, is useful to sufficiently optimize secure, hold, and maintain the limb of a patient 101 (e.g. leg, knee, ankle, etc.) in a desired position against displacement and pressure forces during a medical procedure to the pressure pad apparatus and system 100 disposed on a support bar of a surgical distractor unit. As shown in FIG. 17, the limb of the patient 101 is set in a desired position on a support or operating (OR) table 102 configured with a positioning system 103, a distractor assembly 104, or both (103 and 104) in a medical procedure 105. Accordingly, the present invention is described in an arthroscopic surgery medical procedure 105 in an embodiment of arthroscopy and/or arthroscopic surgery for the repair of the joint (i.e. on the knee as shown in FIGS. 17 and 18) using the pressure pad apparatus and system 100, illustrated in FIGS. 1-16. As shown in FIGS. 17 and 18, the pressure pad apparatus and system is configured to work with a positioner 103 and/or distractor 104 such as the De Mayo Universal Distractor® for the knee and ankle surgical procedures involved in the finite joint distraction of the surgical site, which is described in U.S. Pat. No. 8,048,082 B1, which disclosure is incorporated by reference in its entirety. Although it should appreciated that the principals of the present invention may be used in other arthroscopic surgery medical procedures commonly used for ankle, hip, wrist, elbow, shoulder problems, whereby the improvements and advantages of the pressure pad apparatus and system 100 may further apply to disadvantages in the prior art for the reduction of injury to the joint, tissues, bones, circulatory and nerve structures.

Referring to FIG. 17, in predetermined arthroscopic surgery on the knee medical procedure 105, the positioning pad and system 100 is attached to the limb support bar of a positioning system 103 that secures and holds the limb of the patient 101 (i.e. upper and lower leg and foot) to prevent movement on the OR table 102, which movements could be disruptive to a medical procedure 105 being performed on the patient 101. A pressure pad apparatus 100 may be utilized with the positioning system 103 so as to hyper-extend the joint (i.e. knee or ankle in this configuration) for the medical procedure as illustrated in FIG. 17. Simply, a limb of the patient 101 is positioned on the pressure pad apparatus 100 that is disposed on the limb support bar or post 107 of the positioning system 103. The pressure pad apparatus is also connected to the distractor assembly 104 used to hyper-extend the knee for the medical procedure as illustrated in FIG. 17.

According to the knee arthroscopic surgery embodiment of the present invention, the pressure pad apparatus and system 100 has advantages in the reduction of injury to the joint, tissues and arterial structures of the popliteal area 106 around the knee joint and its tissues, bones, circulatory and nerve structures when distracting a knee for a surgical operation as shown in FIG. 17. In this embodiment, the popliteal area 106 of the patient 101 is protected from injury by the cooperation of the pad assembly 130 with the cage assembly 110. The spring lever 125 of the pad assembly, system and kit 100 is configured to provide a sterile protector pad with a broad distribution of pressure thereby resisting crushing, tearing at the opening 114, or otherwise slipping, sliding, rotating and/or rolling off of the support bar according to an embodiment of the present invention.

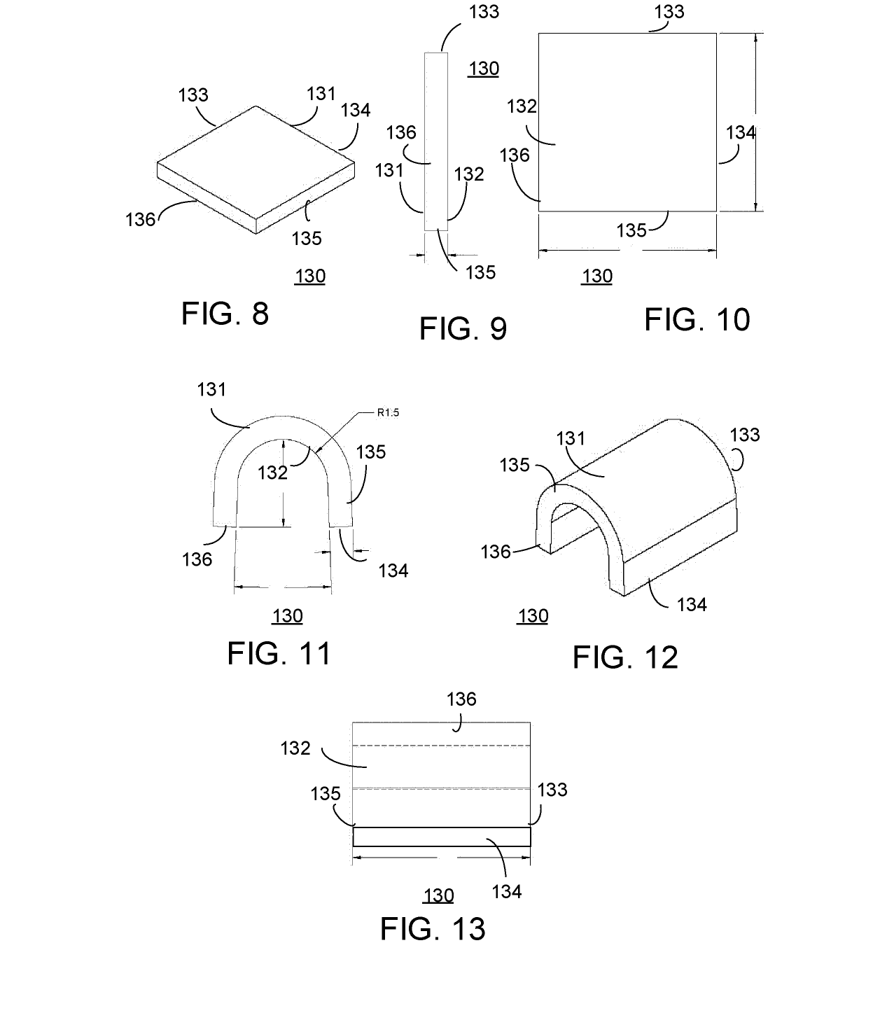

As is illustrated in FIGS. 1-18, the pressure pad apparatus and system 100 comprises of a cage assembly 110 and a pad assembly 130. As is illustrated in FIGS. 8-13, the pad assembly 130 of the pressure pad apparatus and system 100 may be configured as a generally rectangular pad. The pad assembly 130 has a top surface 131, bottom surface 132, and sides 133, 134, 135 and 136. It is to be appreciated that the top surface and the bottom surface may be the same and interchangeable. For ease of the description, the top surface 131 is disposed adjacent the patient's 101 limb near the joint to be extended such as, for example, in the popliteal area 106. The bottom surface 132 the pad assembly 130 is affixed by adhesive to the outer surface 117 on the enclosure 111 of cage assembly 100. The pad assembly 130 may be formed from suitable foam and cell structure materials that can be sterilized such as, for example, polyurethane-based foam. The enclosure or cage assembly 110 is configured with one or more ridges to sufficiently optimize a material strength to the arcuate side of the enclosure so as to not crush under pressure from the surgical distractor unit and/or the weight of the patient's limb of the patient when the sterile cage assembly 110 and pad assembly 130 support a limb of a patient as the support bar of the distractor unit positions the pad assembly 130 in abutment with a popliteal area of a patient's knee joint during a medical procedure.

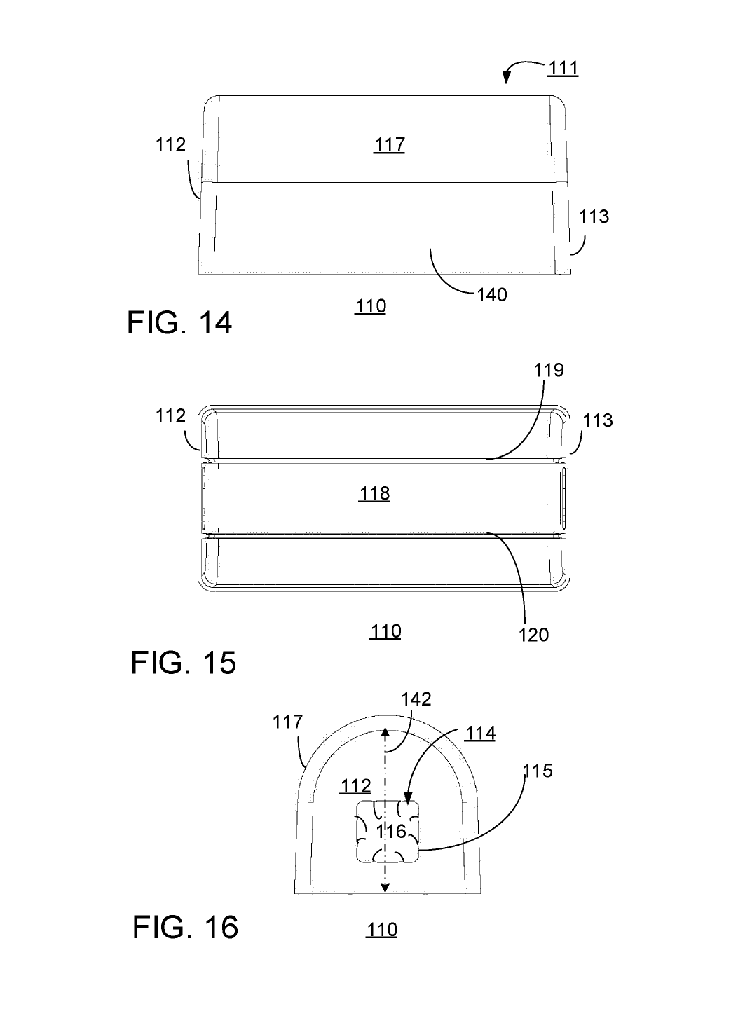

As is illustrated in FIGS. 14-16 the cage assembly 110 comprises an enclosure 111 having a general U-shape and side portions 112 and 113 which may have an opening 114 with a plurality of protrusions or nibs 115 configured to receive the post 107 of a positioning system 103. The enclosure 111 may be formed from plastic materials of suitable strength and durability such as, for example, poly-ester based plastics such as polyethylene terephthalate (PET) and other extruded or molded plastics. The strength of the plastic is required to not crush under pressure from the positioning system 104 such as under the weight of a limb of the patient 101. Moreover, strength of the plastic is required to not tear from pressure from the use of the distractor assembly 104. The durability of the plastic functions to withstand conditions and factors in the operating room, medical procedure and sterilization therefore (i.e. high temperatures for proscribed periods of time).

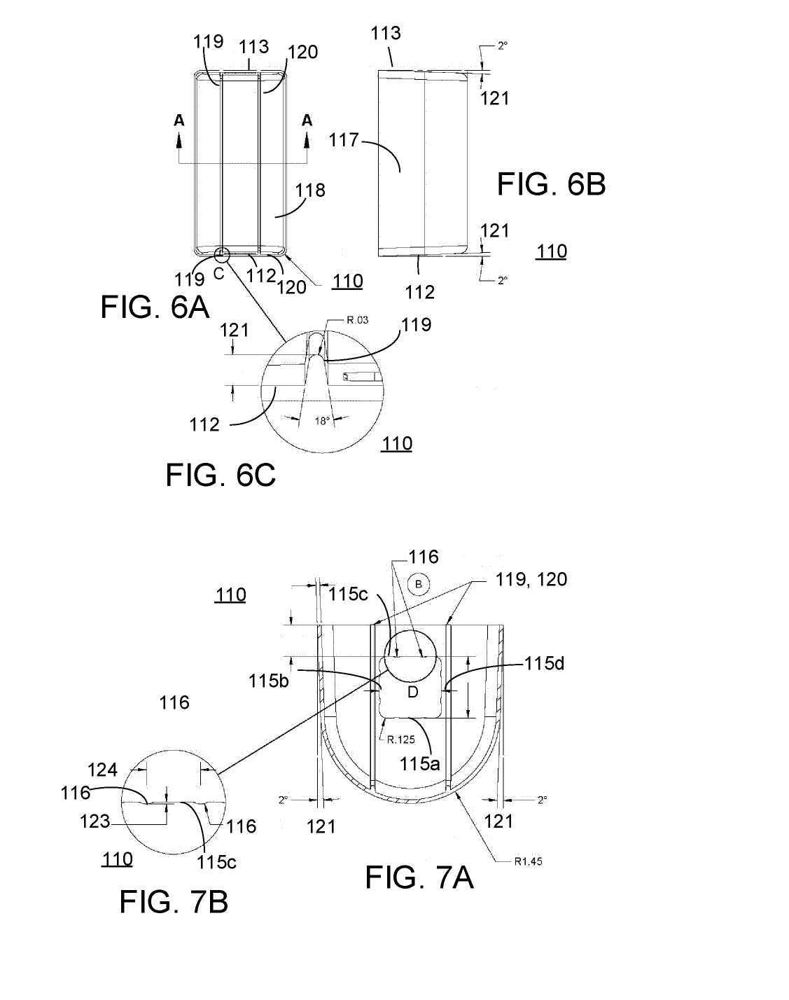

As is illustrated in FIGS. 6A-6C, 7A-7B, 14-16, the enclosure 111 may be formed with side portions 112 and 113 and an arcuate side 140 having an outer surface 117 and an inner surface 118. The outer surface 117 of the arcuate side 140 may be formed generally smooth and in a general U-shape so as to secure the pad assembly 130 thereto such as, for example, using adhesives and the like. The inner surface 118 may be formed with ridges 119 and 120 to provide strength to the arcuate side 140 of the enclosure 111 so as to not crush under pressure from the positioning system 104 such as under the weight of a limb of the patient 101. The one or more ridges 119 and 120 are disposed along the longitudinal axis of the enclosure running along both the side portions 112, 113 and arcuate side 140 (e.g. along the inner surface 118). The one or more ridges 119, 120 also are disposed adjacent the opening 114, i.e. on either side as illustrated in FIGS. 5, 6A-6C and 7A. The design and placement of the ridges 119 and 120 functions (1) strengthen the area of the arcuate side 140 disposed under the limb of the patient 101 to increase load distribution and resist collapse; and (2) to resist tearing of the opening 114 by the post 107 when stressed by the pressure of the distractor assembly 104 pushing the assembly 100 against the limb of the patient 101 as occurs in the medical procedure 105 (e.g. to set and hold the limb in a surgical operation). As shown in FIG. 6C, the one or more ridges 119, 120 are configured to be molded with a predetermined height 122 and shape e.g. approximately at an 18 degree spread at the base with a radius of 0.03. In this manner, the ridges 119 and 120 are configured to reduce weight and cost by using less material for the enclosure 111.

Referring to FIGS. 2 and 6B the side portions 112 and 113 are configured with a predetermined angle 121 may be formed angled (e.g. approximately 2 degrees). As illustrated in FIGS. 7A and 7B, the side portions 112, 113 also may have an opening 114 formed therein. As illustrated in FIGS. 6A-6B, 7A-7C, the opening 114 has nibs 116 formed on an inner edge 115 in a predetermined dimension 123 and spaced apart a predetermined distance 124. The opening 114 is dimensioned to receive the post 107 of the positioning system 103. The nibs 116 are formed on inner edge 115 of the opening 114 and further may be designated edges 115a, 115b, 115c, and 115d as illustrated in FIG. 7A.

The side portions 112, 113 are disposed at a predetermined angle 121 of approximately between 1 and 5 degrees measured from an apex of the u-shape of the arcuate side 140 to a lower edge of one or more of the side portions 112, 113 generally shown herein as 142 in FIG. 1. The edges 115a and 115c and the angle of the outer surfaces 112 and 113 (e.g. preferably 2 degree displacement angle from apex (top) to side edge (bottom)) cooperate to form a spring lever 125 suitable for locking the assembly to the support bar, e.g. of a surgical distraction system. The edges 115b and 115d also provide friction fit on post 107. The spring lever 125 of the edges 115a and 115c uses spaces apart and displaces the nibs 116 on surface 115a and nibs 116 on surface 115c such that when stress is applied by the distractor assembly 104 these nibs 116 create a stronger hold on the support bar or post 107. The spring lever 125 provides a broad distribution of pressure on the protector pad so as not to slip, rotate or roll according to an embodiment of the present invention.

As illustrated in FIGS. 7A and 7B, the nibs 116 are configured to be molded with a predetermined height 122 and shape into the side portions 112, 113. For example, as shown in FIG. 7B, the nibs 116 are formed in a predetermined dimension 123 and spaced apart a predetermined distance 124. The predetermined dimension 123 of the nibs 116 functions to maintain the position of the pressure pad apparatus and system 100 on the support bar or post 107. The nibs 116 improve the friction with the post 107 when the distractor assembly 104 increases the load on the limb of the patient 101, which is an improvement over the prior art. For example, problems of slippage and not holding and maintaining the position on the post 107 occur in prior art systems, for example, when large limbs are operated on in the medical procedure 105.

While certain configurations of structures have been illustrated for the purposes of presenting the basic structures of the present invention, one of ordinary skill in the art will appreciate that other variations are possible which would still fall within the scope of the appended claims. Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An assembly for supporting a limb of a patient during a medical procedure configured to be received by a support bar of a surgical distractor unit for separating anatomical portions of the patient's limb during the medical procedure, said assembly comprising:
an enclosure configured with side portions, an opening formed in each of said side portions adapted to receive slidably by the support bar, and an arcuate side connected to said side portions formed in a generally u-shape, said enclosure is configured with one or more ridges to sufficiently optimize a spring lever under pressure from the surgical distractor unit; and
a pad disposed on said arcuate side of said enclosure.

2. The assembly of claim 1, wherein said enclosure and pad may be sterilized for supporting a limb of a patient during a medical procedure.

3. The assembly of claim 1, wherein said enclosure is configured with one or more ridges to said arcuate side of said enclosure to sufficiently optimize a material strength under pressure from the surgical distractor unit and/or the weight of the patient's limb of the patient.

4. The assembly of claim 3, wherein said enclosure is configured with said ridges disposed along the longitudinal axis of said enclosure between said side portions and said arcuate side.

5. The assembly of claim 1, wherein said side portions are disposed at a predetermined angle of approximately between 1 and 5 degrees from an apex of the u-shape of said arcuate side to a lower edge of said side portions.

6. The assembly of claim 1, wherein said sterile pad being affixed to an outer surface of said enclosure thereto using adhesives.

7. The assembly of claim 1, wherein said sterile pad may be formed from suitable foam and cell structure materials that can be sterilized including polyurethane-based foam.

8. The assembly of claim 1, wherein said enclosure is configured with one or more ridges of said enclosure and said arcuate side to sufficiently optimize a material strength of said assembly when placed under pressure from the surgical distractor unit.

9. The assembly of claim 1, wherein said enclosure is configured with one or more ridges of said enclosure and said arcuate side to sufficiently optimize a material strength of said assembly to support the weight of the patient's limb.

10. A system for supporting a limb of a patient during a medical procedure, said system comprising:
a surgical distractor unit for separating a pair of adjacent bones within a patient's joint during surgery without insertion within said patient comprising an outer cylinder arranged between a support bar at one end to be positioned adjacent the patient's limb and a base support bar at an opposite end thereof for attaching for said surgical distractor unit to a support base, said surgical distractor unit configured to move said support bar relative to said base support bar to control a separation distance between said patient's bones, joints and/or tissues before, during and after surgery thereon;
a cage assembly configured to be received by said support bar so as to abut a patient's joint, said cage assembly comprising:
an enclosure configured with side portions, an opening formed in each of said side portions adapted to receive slidably by said support bar, and an arcuate side connected to said side portions formed in a generally u-shape, said enclosure is configured with one or more ridges to sufficiently optimize a material strength to said arcuate side of said enclosure so as to not crush under pressure from the surgical distractor unit and/or the weight of the patient's limb of the patient, and said opening configured with one or more nibs disposed on an edge of said opening, said nibs configured to sufficiently optimize holding of said enclosure on said support bar in a desired position against pressure forces during a medical procedure; and
a pad disposed on said arcuate side of said enclosure.

11. The system of claim 10, wherein said support bar positions said pad assembly in abutment with a popliteal area of a patient's knee joint.

12. The system of claim 10, wherein said sterile pressure pad being affixed to said gel base portion from the group of a staple, stitch, glue, adhesive, or other fastener.

13. The system of claim 10, wherein said base support bar includes means for attachment to an operating table side rail or a patient limb support device.

14. The system of claim 10, wherein said enclosure and pad are sterile for supporting a limb of a patient during a medical procedure.

15. The system of claim 10, wherein said enclosure is configured with one or more ridges to sufficiently optimize a material strength to said arcuate side of said enclosure so as to not crush under pressure from the surgical distractor unit and/or the weight of the patient's limb of the patient.

16. The system of claim IS, wherein said enclosure is configured with said ridges disposed along the longitudinal axis of said enclosure between said side portions and said arcuate side.

17. The system of claim 10, wherein said side portions are disposed at a predetermined angle of approximately between 1 and 5 degrees from an apex of the u-shape of said arcuate side to a lower edge of said side portions.

18. The system of claim 10, wherein said sterile pad being affixed to an outer surface of said enclosure thereto using adhesives.

19. The system of claim 10, wherein said sterile pad may be formed from suitable foam and cell structure materials that can be sterilized including polyurethane-based foam.

20. A sterile pressure pad kit for supporting a limb of a patient during a medical procedure, said patient anchoring system comprising:
a cage assembly configured to be received by said support bar so as to abut a patient's joint, said cage assembly comprising:
an enclosure configured with side portions, an opening formed in each of said side portions adapted to receive slidably by said support bar, and an arcuate side connected to said side portions formed in a generally u-shape, said enclosure is configured with one or more ridges to sufficiently optimize a material strength to said arcuate side of said enclosure so as to not crush under pressure from the surgical distractor unit and/or the weight of the patient's limb of the patient, and said opening configured with one or more nibs disposed on an edge of said opening, said nibs configured to sufficiently optimize holding of said enclosure on said support bar in a desired position against pressure forces during a medical procedure; and
a pad disposed on said arcuate side of said enclosure wherein said pad adapted to be in abutment with a popliteal area of a patient's knee joint.
